(19) United States
(12) Patent Application Publication
(10) Pub. No.: US 2021/0299320 A1
(43) Pub. Date: Sep. 30, 2021

(54) Safe Antiviral Air-Filtering Lighting Device

(71) Applicant: Aleddra Inc., Renton, WA (US)

(72) Inventors: Chia-Yiu Maa, Bellevue, WA (US); Chun-Te Yu, Bellevue, WA (US)

(21) Appl. No.: 17/147,783

(22) Filed: Jan. 13, 2021

Related U.S. Application Data
(63) Continuation-in-part of application No. 16/836,570, filed on Mar. 31, 2020, now Pat. No. 10,921,004.

(51) Int. Cl. A61L 9/20; B01J 21/06; B01J 23/52; B01J 23/50; B01J 35/00; F21V 23/00; F21V 33/00

(52) U.S. Cl. CPC A61L 9/205; B01J 21/063; B01J 23/52; B01J 23/50; B01J 35/004; F21Y 2115/10; F21V 33/0068; A61L 2209/14; A61L 2209/12; A61L 2209/11; F21V 23/009

(57) ABSTRACT

A lighting device that comprises a housing, a first light source, a second light source, an air filter, an airway, and an air circulation mechanism for each airway. The first light source contributes to the light output of the device, whereas the second light source is responsible for germicidal irradiation and in some embodiments with the help of a photocatalyst coated air filter. The airway has an air inlet and an air outlet. The air circulation mechanism sucks an ambient air through the air inlet, forces the air through the air filter, and releases the air through the air outlet. The air filter traps airborne particles. The UV dosage emitted out of the lighting device does not exceed UV threshold limit value (TLV) dosage defined by American Conference of Governmental Industrial Hygienists (ACGIH).

The present disclosure is part of a Continuation-in-Part (CIP) of U.S. patent application Ser. No. 16/836,570, filed 31 Mar. 2020, the content of which being incorporated by reference in its entirety.

### BACKGROUND

### Technical Field

### Description of Related Art

In U.S. patent application Ser. No. 16/836,570, an antiviral air-filtering lighting device was introduced. The device includes a housing, a first light source, a second light source, an airway, an air filter, and an air circulation mechanism corresponding to the airway. The housing houses the first light source, the second light source, the airway, and the air filter. The first light source emits predominantly visible light (>400 nm) and accounts for 100% of a light output of the lighting device. The second light source is concealed inside the housing, has no contribution to the light output of the lighting device, and emits predominantly ultraviolet (UV) light (<400 nm). The airway has an air inlet and an air outlet. The air circulation mechanism sucks an ambient air through the air inlet, forces the air through the air filter, and releases the air through the air outlet. The air filter traps airborne microbials on a surface thereof and has an antiviral photocatalytic coating on a surface thereof. The second light source is positioned adjacent to the air filter and activates a photocatalyst material in the antiviral photocatalytic coating. The airborne microbials trapped by the air filter are decomposed by the activated photocatalyst material in the antiviral photocatalytic coating.

After further examination of the design taught in U.S. patent application Ser. No. 16/836,570, there are some areas of enhancement being identified from the safety perspective. Firstly, it is taught in U.S. patent application Ser. No. 16/836,570 that the second light source is concealed inside the housing since it is a UV light source for safety reason. American Conference of Governmental Industrial Hygienists (ACGIH) has published a UV Safety Guidelines as shown in FIG. 1 (ACGIH ISBN: 0-9367-12-99-6). It shows the UV Threshold Limit Values (TLVs), which is the maximum allowable dosage (in mJ/cm²) for each UV wavelength over an 8-hour period. For example, the TLV for 222 nm wavelength is set to 22 mJ/cm². The concealment restriction of the second light source may be relaxed if the UV emission coming out of the fixture does not exceed the UV TLV dosage defined by ACGIH.

Secondly, it is known that UV light source, especially the ultraviolet-C(UVC) light source has germicidal irradiation effect on airborne microbial, and it does not need a photocatalyst to achieve the killing of microbial, though a photocatalyst could enhance the killing of microbial. This leads to the possibility of using a sufficiently strong UVC light source, with or without a photocatalyst coating on the air filter, in the antiviral air-filtering lighting device. The condition of a UVC light source being sufficiently strong for an antiviral air-filtering lighting device may be defined by two criteria. The first criterion is that the UVC light source must emit at least fifty times of the UV TLV dosage defined by ACGIH at zero distance. This is to ensure the UVC light source is strong enough out of the gate. This criterion on the UV dosage of the second light source is necessary but insufficient.

Considering the same UVC light source being disposed in the center of two different airways, one airway has a width of 20 cm for the cross section of the airway containing the UVC light source, and the other airway has a width of 100 cm for the cross section of the airway containing the UVC light source. With the first airway, the farthest distance of the UVC light source to the wall of the airway may be 10 cm, whereas with the second airway, the farthest distance of the UVC light source to the wall of the airway may be 50 cm. The UVC dosage received at 50 cm is much less than the UVC dosage received at 10 cm. Therefore, it is critical to establish another UVC dosage criterion such that there is sufficient UVC dosage everywhere in a cross section of the airway (perpendicular to the direction of air flow) containing the UVC light source. This would ensure when air passes a cross section of the airway (perpendicular to the direction of air flow) containing the UVC light source, it will always receive sufficient UVC dosage at any location on that cross section. The larger the dimension of the cross section of the airway containing the UVC light source, the higher the UVC radiant power from the UVC light source is required for ensure the farthest location of this cross section would still receive sufficient UVC dosage.

### SUMMARY

With different embodiments, the present disclosure transforms a regular lighting equipment to an antiviral air-filtering equipment, thereby bringing wellness lighting to the daily life of a user.

In one aspect, the lighting device comprises a housing, a first light source, a second light source, an air filter, an airway, and an air circulation mechanism corresponding to the airway. The housing houses the first light source, the second light source, the air filter, and the airway. The first light source emits predominantly visible light (>400 nm) and accounts for at least 95% of the light output of the lighting device. The second light source is disposed inside the airway and contribute less than 5% of the light output of the lighting device, and the second light source emits predominantly an ultraviolet (UV) light (<400 mn). The condition on the second light source contributes less than 5% of the light output refers to the light leaking out of the airway and becoming part of the light output of the lighting device. This condition does not change the fact the second light source contributes 100% of the UV light inside the airway. The airway has an air inlet and an air outlet. The air circulation mechanism sucks an ambient air through the air inlet, forces the air through the air filter, and releases the air through the air outlet. The air filter traps airborne particles carried in the air. The UV dosage emitted out of the lighting device does not exceed a UV threshold limit value (TLV) dosage defined by American Conference of Governmental Industrial Hygienists (ACGIH). It is not necessary for the second light source to be sealed in the airway or the housing, so long it does not contribute more than 5% of the light output of the light device. However, given that the second light source is a UV light source, it is critical to establish a condition that the lighting device does not leak out dangerous amount of UV dosage into the environment. When complying with the UV TLV restriction defined by ACGIH, the light device would be safe to operate in an environment with occupants.

In some embodiments, the UV dosage emitted out of the second light source at a UV wavelength at zero distance is at least fifty times of a UV TLV dosage defined by ACGIH for the wavelength. This is to ensure the second light source could emit sufficient UV dosage for germicidal irradiation, measured perhaps on a peak UV wavelength of the second light source. Different UV wavelengths have different effects on killing bacteria and viruses. It is noted that from FIG. 1, each wavelength has a different TLV dosage limit. This condition on one (peak) wavelength simplifies the discussion and facilitates implementation.

In some embodiments, the UV dosage at a UV wavelength received everywhere in a cross section of the airway (perpendicular to the direction of the air flow) containing the second light source is at least ten times of a UV TLV dosage defined by ACGIH for the wavelength. This is to ensure the air will always receive sufficient UV exposure even when the air passes through the farthest corner of the airway cross section containing the second light source. Without this condition, the second light source may not be able to cover the entire cross section of the airway where it is disposed with sufficient irradiance (in unit of mW/cm²).

In some embodiments, the linear second light source emitting predominantly a UVC light (190-280 nm), i.e., the second light source is a UVC light source. In this case, the second light source may perform germicidal irradiation with and without the assistance of a photocatalyst coated air filter.

In some embodiments, the air filter has an antiviral photocatalytic coating on a surface thereof. The second light source, which is not used for lighting purpose, is positioned adjacent to the air filter for activating the photocatalyst material in the antiviral photocatalytic coating. It is not required for the second light source to be positioned inside the airway. It is only necessary for the second light source to be adjacent to the air filter so that it can effectively activate the photocatalyst material in the antiviral photocatalytic coating on the air filter. When airborne microbials are trapped by the air filter, and the activated photocatalyst material in the antiviral photocatalytic coating would kill and decompose the trapped microbials.

If using visible-light activate-able photocatalyst material, a visible light source can be used as the second light source for trigger the photocatalytic activity. Therefore, in some embodiments, the photocatalyst material in the antiviral photocatalytic coating on the air filter may be activate-able by visible light (>400 nm).

In some embodiments, the photocatalyst material in the antiviral photocatalytic coating contains one type of material, titanium oxide (TiO₂). It is also common to use TiO₂ with another metal for the metal may help TiO₂ absorb the energy in the visible light range. Therefore, in some embodiments, the photocatalyst material in the antiviral photocatalytic coating contains titanium oxide (TiO₂) as the primary photocatalyst and an active metal ingredient such as silver, gold, copper, zinc, nickel, or a combination thereof, as the secondary photocatalyst. Liu's teaching in U.S. Pat. No. 9,522,384 demonstrates the use of TiO₂ as the primary photocatalyst and silver as the secondary photocatalyst.

The titanium dioxide is classified as a semiconducting photocatalyst. Recently, technology breakthrough has demonstrated that noble metal nanoparticles such as gold (Au) and silver (Ag) can are a class of efficient photocatalysts working by mechanisms distinct from those of semiconducting photocatalysts (https://pubs.rsc.org/en/content/articlelanding/2013/gc/c3gc40450a#!divAbstract). The present disclosure is not limited to the use of semiconducting photocatalysts such as TiO₂ only. The metal-based photocatalysts would work just as well. Therefore, in some embodiments, the photocatalyst material in the antiviral photocatalytic coating contains a noble metal nanoparticle such as gold (Au) or sliver (Ag) as the main photocatalyst.

It is known that activated carbon can absorb and remove gases and bad odors. In some embodiments, the air filter contains activated carbon.

In some embodiments, the air circulation mechanism is a fan positioned in the airway. However, other air circulation mechanism, such as an external HVAC system may also be used as the air circulation mechanism of the present disclosure so long it may move effectively the ambient air through the airway.

In some embodiments, the first light source may comprise white light LEDs each emitting predominantly visible light (>400 nm). In some embodiments, the second light source may comprise UV LEDs each emitting predominantly UV light (<400 nm).

In some embodiments, the first light source may further comprise a third light source and a fourth light source. Both emit predominantly visible light (>400 nm), and the color temperature of the third light source is higher than the color temperature of the fourth light source. The use of the third light source with a higher color temperature is for providing a higher circadian stimulus, which is desirable for some lighting applications for mimicking the daylight. Similarly, the use of the fourth light source with a lower color is to provide a lower circadian stimulus for nighttime. Moreover, in some embodiments, the color temperature of the first light source may be tunable via a controller by mixing the combination ratio of color temperatures of the third light source and the fourth light source. This color tuning may be done manually, or it may be done automatically according to a circadian schedule stored in a memory module. The circadian schedule will transition the color temperature of the first light source from warm white (2700K) to cold white (6500K) at dawn and revert the color temperature back to warm white at sunset, thus emulating color transition of the sunlight.

### BRIEF DESCRIPTION OF THE DRAWINGS

The accompanying drawings are included to aid further understanding of the present disclosure, and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 The Threshold Limit Values (dosage) according to ACGIH UV Safety Guidelines.

FIG. 2a schematically depicts a cross-section diagram of an LED screw-in lamp as an embodiment of the present disclosure.

FIG. 2b schematically depicts a diagram of the screw-in lamp from another perspective.

FIG. 2c schematically depicts a diagram of the screw-in lamp from yet another perspective.

FIG. 2d schematically depicts the air filter and its antiviral photocatalytic coating of the lamp, and the trapped microbials.

FIG. 3a schematically depicts an exterior view of an LED troffer fixture.

FIG. 3b schematically depicts a look-through view of the troffer.

FIG. 3c schematically depicts a look-through view of one of the two airways of the troffer.

FIG. 3d schematically depicts the air filter and its activated carbon coating of the troffer, and the trapped microbials.

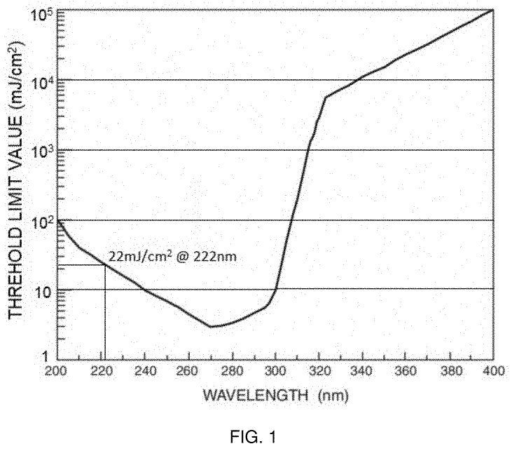

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of lighting devices having different form factors.

The present disclosure discloses a lighting device that comprises a housing, a first light source, a second light source, an air filter, an airway, and an air circulation mechanism for each airway. The first light source contributes to the light output of the device, whereas the second light source is responsible for germicidal irradiation and in some embodiments with the help of a photocatalyst coated air filter. The airway has an air inlet and an air outlet. The air circulation mechanism sucks an ambient air through the air inlet, forces the air through the air filter, and releases the air through the air outlet. The air filter traps airborne particles carried in the air. The UV dosage emitted out of the lighting device does not exceed UV threshold limit value (TLV) dosage defined by American Conference of Governmental Industrial Hygienists (ACGIH).

### Example Implementations

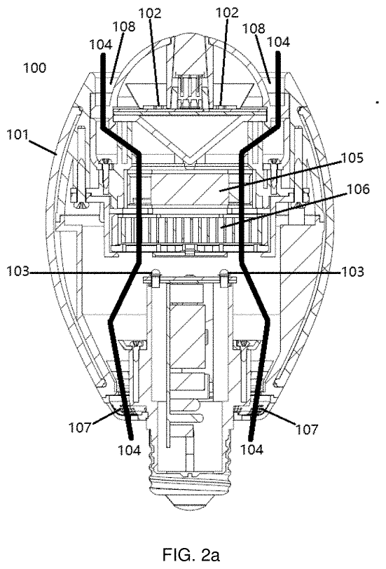

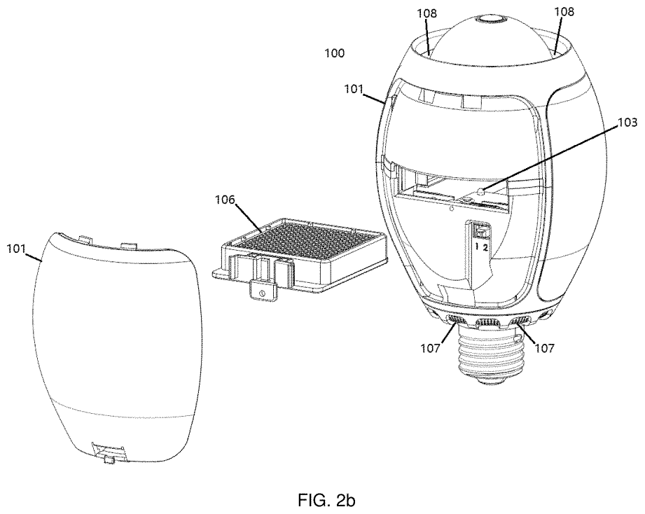

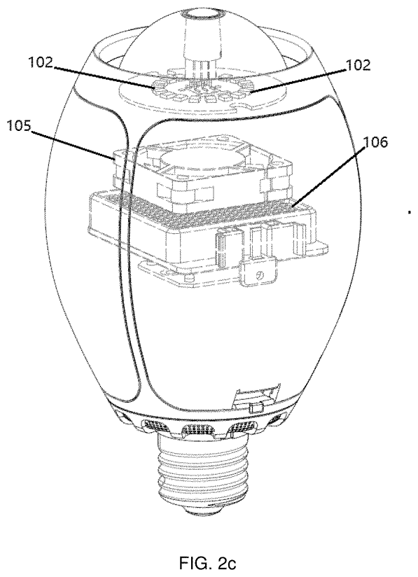

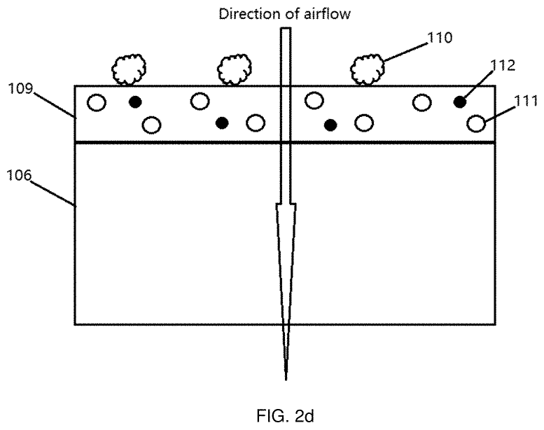

FIG. 2a-2c show an embodiment of the lighting device of the present disclosure in a form of an LED screw-in lamp 100. This lamp has a housing 101, the first light source 102, the second light source 103, one airway 104, and one fan 105 and one air filter 106. The first light source 102 comprises multiple LEDs on top of the lamp emitting predominantly white light in the >400 nm wavelength range, and accounts for 100% light output of the lighting device. The second light source 103 comprises multiple UV LEDs and are concealed inside the housing and has no contribution to the light output of the lighting device. The airway 104 has an air inlet 107 and an air outlet 108. The fan 105 and the air filter 106 are positioned inside the airway 104. The fan 105 sucks the ambient air through the air inlet 107, forces the air through the air filter 106, and releases the air through the air outlet 108. The air filter 106 has antiviral photocatalytic coating 109 on its surface. The second light source 103 is positioned adjacent to the air filter 106 for activating the photocatalyst material in the antiviral photocatalytic coating 109. In this embodiment, the second light source 103 is inside the airway 104. Since the second light source 103 is completely concealed inside the housing, there would not be any UV light leaks out of this lamp 100, thus meeting a UV threshold limit value (TLV) dosage defined by American Conference of Governmental Industrial Hygienists (ACGIH).

In FIG. 2d, as the air passes through the air filter 106, the airborne microbials 110 are trapped on the surface of the air filter. The photocatalyst material in the antiviral photocatalytic coating 109 activated by the second light source 103 kills and decomposes the trapped microbials 110. The photocatalyst material contains nano anatase-type TiO₂ 111 as its primary photocatalyst and nano silver 112 as its secondary photocatalyst. When using TiO₂ and nano silver as photocatalyst, visible light LEDs can be used as the second light source 103 and still achieve adequate photocatalytic effect. It is also foreseeable to use metals other than nano silver as the secondary photocatalyst. Moreover, it is anticipated to use noble metal nanoparticle such as gold (Au) and silver (Ag) as the main photocatalyst in lieu of a semiconducting photocatalyst TiO₂.

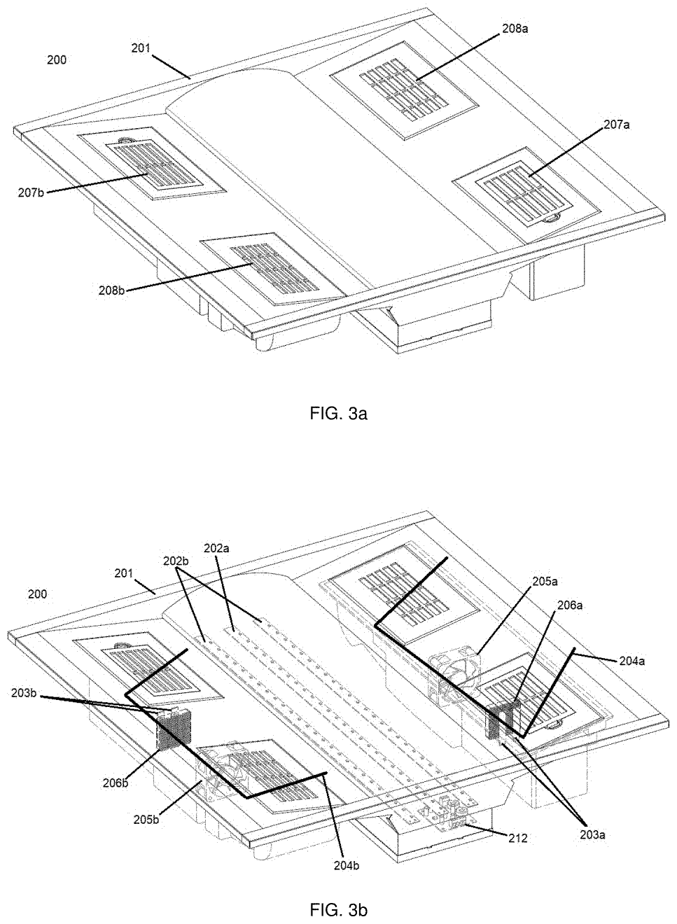

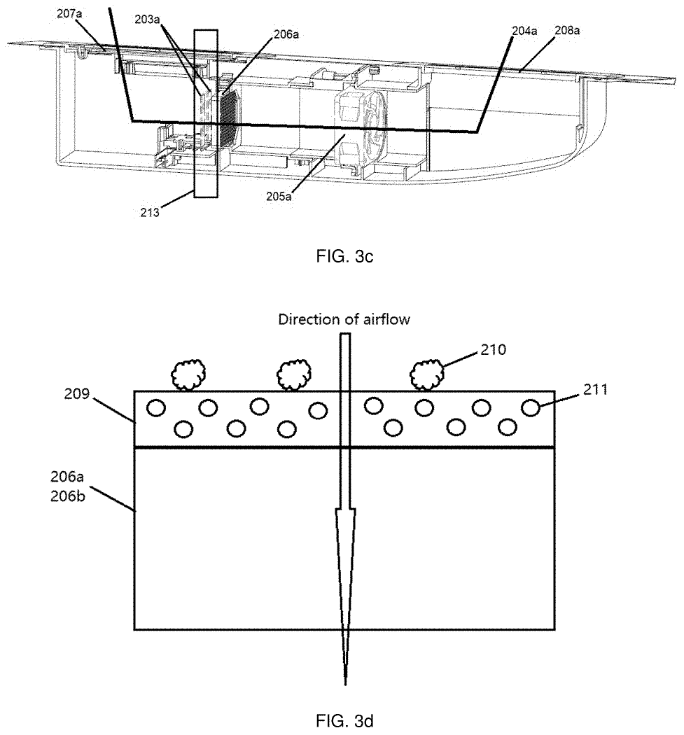

FIGS. 3a, 3b, and 3c show an embodiment of the lighting device of the present disclosure in a form of an LED troffer fixture 200. This troffer has a housing 201, the first light source 202a,202b, the second light source 203a,203b, two fans 205a,205b, two air filters 206a,206b, and two airways 204a,204b. The first light source comprises three rows of LEDs on three PCBs. Out of the three rows of LEDs, one row is 2700K LEDs 202a and the other two rows are 6500K LED 202b. The 2700K LEDs produce a lesser circadian stimulus and is more suitable for nighttime lighting, whereas the 6500K LED produce a higher circadian stimulus and is more suitable for daytime use. The combined light output of 2700K and 6500K LEDs sets the total light output of the lighting device. Since they each emits predominantly visible light in the >400 nm wavelength range, their combined light is also in the >400 nm wavelength range. A controller 212 is used to color-tune the light output of the light device by changing the mixing ratio of the light output of the 2700K LEDs 202a and the 6500K LEDs 202b. Though not shown, a memory module may be used to store a circadian schedule in the controller 212. The controller 212 can thus color-tune the light output of the first light source automatically according the circadian schedule stored in the memory module. The circadian schedule will transition the color temperature of the first light source from warm white (2700K) to cold white (6500K) at dawn and revert the color temperature back to warm white at sunset, thus emulating color transition of the sunlight.

The construction of the two airways 204a and 204b are the same, therefore the description below is on the airway 204a. The second light source 203a in the airway 204a comprises multiple UVC LEDs and has no contribution to the light output of the lighting device. However, because the second light source 203a is not completely conceal, its UV light may still leak out of the airway 204a. The construction of the airway 204a would ensure the UV dosage leaking out of the airway 204a do not exceed a UV TLV defined by ACGIH, e.g., by using UV light absorbing coating on the inside wall of the airway 204a.

The airway 204a has an air inlet 207a and an air outlet 208a. The fan 205a and the air filter 206a are positioned inside the airway 204a. As the fan 205a forces the air through the airway 204a, the airborne microbials 210 are trapped on the surface of the air filter 206a. The surface air filter 206a is coated with an activated carbon 211 coating 209 for removing the gases and the bad odors in the air. The second light source 203a, being UVC LEDs, emits more than fifty times of the UV TLV dosage defined by ACGIH, and provides more than ten time the UV TLV dosage defined by ACGIH anywhere in the cross section 213 of the airway 204a that contains the second light source 203a. The second light source 203a performs germicidal irradiation on the microbials trapped on the surface of the air filter 206a as well as the microbials passing through the cross section 213. Note that the germicidal irradiation of microbials by UVC is not instantaneous. It is highly likely a microbial may not receive sufficient UVC dosage as it passes through the cross section 213 and thus may not be completely killed (though damaged). However, this microbial may still be trapped by the air filter 206a, so that the UVC light source 203a has plenty of time to complete the killing of the microbial.

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A lighting device comprises:
a housing;
a first light source;
a second light source;
an air filter;
an airway;
an air circulation mechanism corresponding to the airway,
wherein, in operation:
the housing houses the first light source, the second light source, the air filter, and the airway,
the first light source emits a visible light with a wavelength greater than 400 nm and accounts for at least 95% of a light output of the lighting device,
the second light source is disposed inside the airway and contributes less than 5% of the light output of the lighting device,
the second light source emits a an ultraviolet (UV) light with a wavelength less than 400 mn,
the airway has an air inlet and an air outlet,
the air circulation mechanism sucks an ambient air through the air inlet, forces the air through the air filter, and releases the air through the air outlet,
the air filter traps airborne particles carried in the air, and
a UV dosage emitted out of the lighting device does not exceed a UV threshold limit value (TLV) dosage defined by American Conference of Governmental Industrial Hygienists (ACGIH).

2. A lighting device of claim 1, wherein a UV dosage emitted out of the second light source at a UV wavelength at zero distance is at least fifty times of a UV TLV dosage defined by the ACGIH for the UV wavelength.

3. A lighting device of claim 1, wherein a UV dosage at a UV wavelength received everywhere in a cross section of the airway containing the second light source is at least ten times of a UV TLV dosage defined by ACGIH for the UV wavelength.

4. A lighting device of claim 1, wherein the second light source emits an ultraviolet-C (UVC) light in a wavelength range of 190-280 nm.

5. A lighting device of claim 1, wherein:
the air filter has an antiviral photocatalytic coating on a surface thereof,
the second light source is positioned adjacent to the air filter and activates a photocatalyst material in the antiviral photocatalytic coating, and
airborne microbials trapped by the air filter are decomposed by the activated photocatalyst material in the antiviral photocatalytic coating.

6. A lighting device of claim 5, wherein the photocatalyst material in the antiviral photocatalytic coating on the air filter is activatable by the visible light.

7. A lighting device of claim 5, wherein the photocatalyst material in the antiviral photocatalytic coating on the air filter contains titanium oxide (TiO₂).

8. A lighting device of claim 5, wherein the photocatalyst material in the antiviral photocatalytic coating on the air filter contains titanium oxide (TiO₂) as a primary photocatalyst and an active metal ingredient as the secondary photocatalyst, and wherein the active metal ingredient comprises silver, gold, copper, zinc, nickel, or a combination thereof.

9. A lighting device of claim 5, wherein the photocatalyst material in the antiviral photocatalytic coating on the air filter contains a noble metal nanoparticle gold (Au) or sliver (Ag) as a main photocatalyst.

10. A lighting device of claim 1, wherein the air filter contains activated carbon.

11. A lighting device of claim 1, wherein the air circulation mechanism comprises a fan positioned in the airway.

12. A lighting device of claim 1, wherein the first light source comprises white-light light emitting diodes (LEDs) each emitting the visible light.

13. A lighting device of claim 1, wherein the second light source comprises UV light emitting diodes (LEDs) each emitting the UV light.

14. A lighting device of claim 1, wherein the first light source further comprises a third light source and a fourth light source, wherein both the third light source and the fourth light source emit the visible light, and wherein a color temperature of the third light source is higher than a color temperature of the fourth light source.

15. A lighting device of claim 14, further comprising:
a controller,
wherein the controller is configured to tune a color temperature of the first light source by mixing a combination ratio of the color temperatures of the third light source and the fourth light source, either manually or automatically, according to a circadian schedule stored in a memory module of the controller.
